(19) United States
(12) Patent Application Publication
(10) Pub. No.: US 2006/0211919 A1
(43) Pub. Date: Sep. 21, 2006

(54) Intra-abdominal medical device and associated method

(75) Inventor: Peter J. Wilk, New York, NY (US)

Correspondence Address: COLEMAN SUDOL SAPONE, P.C., 714 COLORADO AVENUE, BRIDGE PORT, CT, 06605-1601, US

(73) Assignee: WILK PATENT. LLC, New York, NY (US)

(21) Appl. No.: 11/369,499

(22) Filed: Mar. 7, 2006

Related U.S. Application Data
(60) Provisional application No. 60/662,981, filed on Mar. 18, 2005.

(51) Int. Cl. A61B 1/32

(52) U.S. Cl. 600/207

(57) ABSTRACT

A surgical device includes a disk made of a flexible sheet material and a balloon. The disk and the balloon define respective apertures that are aligned with one another to define a hole through the device. The balloon is attached to the disk and has an inflation port for enabling an introduction of a fluid into the balloon to expand same from a collapsed configuration to an inflated configuration.

### CROSS-REFERENCE TO RELATED APPLICATION

This application claims the benefit of U.S. Provisional Patent Application No. 60/662,981 filed Mar. 18, 2005.

### BACKGROUND OF THE INVENTION

This invention relates to medical procedures carried out without the formation of an incision in a skin surface of the patient.

Such procedures are described in U.S. Pat. Nos. 5,297,536 and 5,458,131.

As described in those patents, a method for use in intra-abdominal surgery comprises the steps of (a) inserting an incising instrument with an elongate shaft through a natural body opening into a natural body cavity of a patient, (b) manipulating the incising instrument from outside the patient to form a perforation in an internal wall of the natural internal body cavity, and (c) inserting a distal end of an elongate surgical instrument through the natural body opening, the natural body cavity and the perforation into an abdominal cavity of the patient upon formation of the perforation. Further steps of the method include (d) inserting a distal end of an endoscope into the abdominal cavity, (e) operating the surgical instrument to perform a surgical operation on an organ in the abdominal cavity, (f) viewing the surgical operation via the endoscope, (g) withdrawing the surgical instrument and the endoscope from the abdominal cavity upon completion of the surgical operation, and (h) closing the perforation.

Visual feedback may be obtained as to position of a distal end of the incising instrument prior to the manipulating thereof to form the perforation. That visual feedback may be obtained via the endoscope or, alternatively, via radiographic or X-ray equipment.

The abdominal cavity may be insufflated prior to the insertion of the distal end of the endoscope into the abdominal cavity. Insufflation may be implemented via a Veress needle inserted through the abdominal wall or through another perforation in the internal wall of the natural body cavity. That other perforation is formed by the Veress needle itself. U.S. Pat. No. 5,209,721 discloses a Veress needle that utilizes ultrasound to detect the presence of an organ along an inner surface of the abdominal wall.

A method in accordance with the disclosures of U.S. Pat. Nos. 5,297,536 and 5,458,131 comprises the steps of (i) inserting an endoscope through a natural body opening into a natural body cavity of a patient, (ii) inserting an endoscopic type incising instrument through the natural body opening into the natural body cavity, (iii) manipulating the incising instrument from outside the patient to form a perforation in an internal wall of the natural internal body cavity, (iv) moving a distal end of the endoscope through the perforation, (v) using the endoscope to visually inspect internal body tissues in an abdominal cavity of the patient, (vi) inserting a distal end of an elongate surgical instrument into the abdominal cavity of the patient, (vii) executing a surgical operation on the internal body tissues by manipulating the surgical instrument from outside the patient, (viii) upon completion of the surgical operation, withdrawing the surgical instrument and the endoscope from the abdominal cavity, (ix) closing the perforation, and (x) withdrawing the endoscope from the natural body cavity.

The surgical procedures of U.S. Pat. Nos. 5,297,536 and 5,458,131 reduces trauma to the individual even more than laparoscopic procedures. Hospital convalescence stays are even shorter. There are some potential problems with the procedures, such as the difficulty in forming a fluid tight closure of the perforation formed in the wall of the hollow internal body organ. Certain intra-abdominal operations cannot be easily performed owing to the necessity or removing large chunks of organic or inorganic material (e.g., entire kidney, gall stones). Some operations can require the simultaneous usage of many different instruments so that space along the selected pathways may be difficult to find.

### OBJECTS OF THE INVENTION

It is an object of the present invention to provide improvements on the afore-described surgical procedures.

It is another object of the present invention to provide a method and/or an associated device for keeping a passageway open in an internal hollow organ.

These and other objects of the present invention will be apparent from the drawings and detailed descriptions herein. While every object of the invention is believed to be attained in at least one embodiment of the invention, there is not necessarily any single embodiment that achieves all of the objects of the invention.

### SUMMARY OF THE INVENTION

A surgical device comprises, in accordance with the present invention, a disk made of a flexible sheet material and a balloon. The disk and the balloon define an aperture extending through the device. The balloon is attached to the disk and has an inflation port for enabling an introduction of a fluid into the balloon to expand same from a collapsed configuration to an inflated configuration.

Pursuant to another feature of the present invention, a valve element is provided on the device for forming a seal about an instrument shaft inserted through the aperture. The valve element may be realized as a resilient annular flange or film material the aperture in the disk and the balloon. Thus, the valve element is attached at least one of the disk and the balloon.

Pursuant to another feature of the present invention, an elongate tube is attached to at least one of the disk and the balloon, the tube extending from a side of the balloon opposite the disk to a side of the disk opposite the balloon. This tube is provided for the introduction of gas to maintain pneumoperitoneum in the abdominal cavity of the patient. Accordingly, the tube is provided at one end with a port element for coupling the tube to a source of pressurized gas. The tube may extend through the aperture in the disk and the balloon.

Pursuant to a further feature of the present invention, the disk can be provided along an edge or periphery with a ring of a resilient material stiffer than the flexible sheet material of the disk. The ring assists in spreading the disk during a deployment procedure and maintaining the disk in an opened configuration against a wall of an internal body organ during an intra-abdominal therapeutic or diagnostic procedure.

Typically, the aperture defined by the disk and the balloon is centrally situated therein. Typically, the disk and the balloon are annular members each surrounding the respective aperture.

In accordance with an additional feature of the present invention, a surface of at least one of the disk and the balloon is provided with a layer of a dormant adhesive substance that activated by the application of a predetermined form of energy.

The disk and the balloon are made of a bioabsorbable biocompatible material.

A surgical method in accordance with the present invention comprises (1) inserting a distal end portion of a surgical instrument through a natural body opening of a patient into a natural body cavity of the patient, (2) using the surgical instrument to form a temporary artificial opening through a wall of an organ defining the natural body cavity, (3) providing a surgical port device comprising a disk made of a flexible sheet material and a balloon that together define an aperture, (4) inserting the port device through the natural body opening into the natural body cavity, and (5) subsequent to the inserting of the port device, disposing the port device in the artificial opening to keep the same open. The disposing of the port device includes inserting one of the disk and the balloon in a collapsed configuration through the artificial opening and expanding each of the disk and the balloon from a collapsed configuration to an expanded configuration so that the wall of the organ is sandwiched between the expanded disk and the expanded balloon and so that the disk and the balloon being connected to one another through the artificial opening. The aperture is aligned with the artificial opening upon the disposing of the port device in the artificial opening. After the disposing of the port device in the artificial opening, inserting a distal end portion of a medical instrument through the natural body opening, the natural body cavity, the aperture and the artificial opening into an internal space inside the patient.

Where the port device includes a valve element, the inserting of the distal end portion of the medical instrument includes engaging an outer surface of the medical instrument with the valve element to form a seal about the medical instrument. The valve element may take the form of a resilient annular flange.

According to another feature of the present invention, the surgical method further comprises introducing a pressurized gas into the internal space via an elongate tube communicating with the internal space via the port device. The tube may extend through the aperture in the port device.

Where the disk is provided along an edge or periphery with a ring of a resilient material stiffer than the flexible sheet material of the disk, the disposing of the port device includes unfolding the ring from a folded configuration.

Where a selected one of the disk and the balloon is provided with a layer of an activatable adhesive, the method further comprises directing a predetermined form of energy towards a surface of the selected one of the disk and the balloon in contact with the wall of the organ to activate the adhesive.

Upon deployment of a port device in accordance with the present invention, either the disk or the balloon is disposed in contact with an inner surface of a hollow organ such as the stomach, the colon, the urinary bladder or the vagina, that communicates with the ambient environment through a natural body opening such as the mouth, the anus, the urethra or the vaginal opening. The other of the disk and the balloon is in contact with an outer surface of the respective hollow organ, that outer surface facing into an internal cavity such as the abdominal cavity.

A surgical kit in accordance with the present invention comprises a surgical instrument having a distal end insertable through a natural body opening of a patient into a natural body cavity of the patient, the surgical instrument being provided with an operative tip utilizable to form a temporary artificial opening through a wall of an organ defining the patient's natural body cavity. The kit further comprises a surgical port device including a disk made of a flexible sheet material and a balloon connected to one another, the disk and the balloon defining an aperture. The port device is insertable through the natural body opening into the natural body cavity and subsequently attachable to the wall of the organ in a region about in the artificial opening to keep the same open. The disk and the balloon are adapted to sandwich the wall of the organ between the disk and the balloon upon an expansion of the disk and the balloon so that the aperture is aligned with the artificial opening upon the disposing of the port device in the artificial opening. The kit optionally includes a medical instrument with a distal end portion through the natural body opening, the natural body cavity, the aperture, and the artificial opening into an internal space inside the patient.

The device and method of the present invention serve to provide support to an inner wall of an internal hollow organ to facilitate the performance of a surgical or diagnostic operation via that hollow organ wall. The present invention makes it easier for a surgeon to locate and identify the artificial opening or aperture formed in the wall of the hollow organ, for purposes of inserting a succession of instruments through the artificial opening or aperture into the internal abdominal space of the patient. Pneumoperitoneum is also maintained.

### BRIEF DESCRIPTION OF THE DRAWINGS

FIG. 1 is a schematic front elevational view of a surgical port device in accordance with the present invention.

FIG. 2 is a schematic side elevational view of the device of FIG. 1.

FIG. 3 is a view similar to FIG. 2, showing the device of FIG. 1 deployed in a patient in a method in accordance with the present invention.

FIG. 4 is a view similar to FIG. 3, showing a modification of the device of FIG. 1 deployed in a patient in a method in accordance with the present invention.

### DETAILED DESCRIPTION

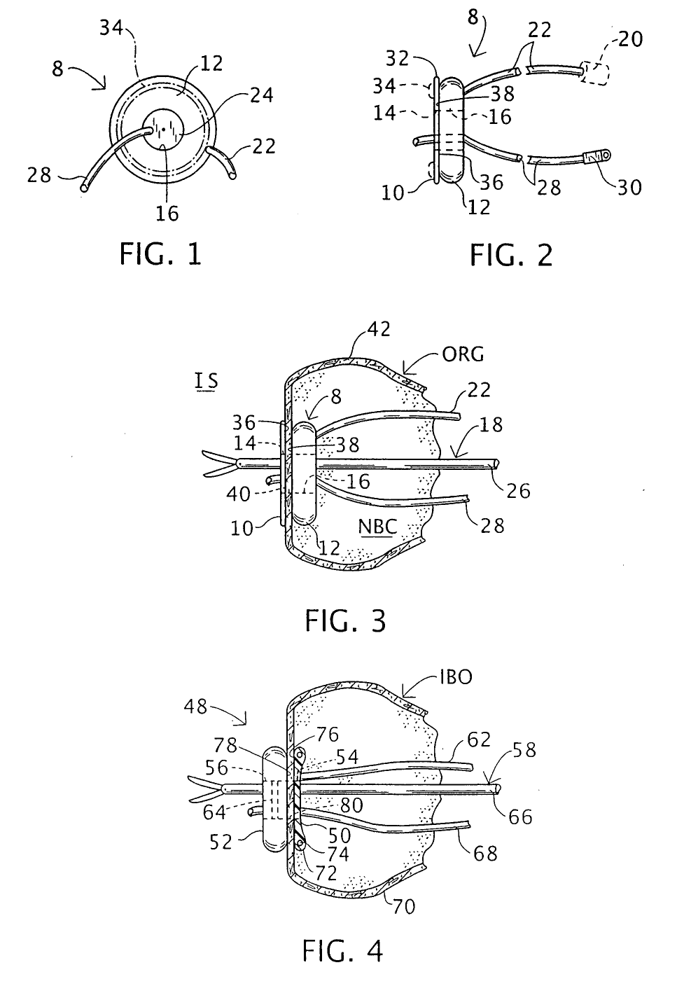

As illustrated in FIGS. 1 and 2, a surgical port device 8 for use in a trans-organ surgical procedure as described in U.S. Pat. Nos. 5,297,536 and 5,458,131 (both incorporated by reference herein) includes a disk 10 made of a flexible sheet material and a balloon 12. Disk 10 and balloon 12 define respective apertures 14 and 16 that are aligned with one another to define a hole for the passage of a medical instrument 18 through the device. Balloon 12 is attached to disk 10 and has an inflation port 20 at the upstream end of an inflation tube 22 for enabling an introduction of a pressurizing fluid into the balloon to expand the balloon from a collapsed insertion configuration to an inflated use configuration.

At least one valve element 24 in the form of a self-sealing membrane or film is provided on the device for forming a seal about the shaft 26 of medical instrument 18 upon insertion thereof through apertures 14 and 16. Valve element or self-sealing membrane 24 may be realized as a resilient annular flange or film material about at least one of the apertures 14 and 16 in the disk 10 and the balloon 12. Thus, valve element 24 is attached at least one of the disk 10 and the balloon 12.

An elongate tube 28 is attached to disk 10 and balloon 12, the tube extending from a side of the balloon opposite the disk to a side of the disk opposite the balloon. In other words, tube 28 traverses the port device. This tube 28 is provided for the introduction of gas (e.g., carbon dioxide) to maintain pneumoperitoneum in the abdominal cavity of a patient during a trans-organ procedure as described in U.S. Pat. Nos. 5,297,536 and 5,458,131. Accordingly, tube 28 is provided at one end with a port element 30 for coupling tube 28 to a source of pressurized gas (not shown). Tube 28 may extend through apertures 14 and 16 in disk 10 and balloon 12.

Disk 10 may be provided along an edge or periphery 32 with a ring 34 of a resilient material stiffer than the flexible sheet material of the disk. Ring 34 assists in spreading disk 10 during a deployment procedure, after a passing of disk 10 in a collapsed form through an artificial opening formed in an internal body organ such as the stomach, colon, vagina, or urinary bladder, and maintaining the disk in an opened configuration against a wall of the internal body organ during an intra-abdominal therapeutic or diagnostic procedure. Alternatively, where ring 34 is omitted, disk 10 is held in an opened configuration by the higher gas pressure in the internal space (abdominal cavity) of the patient in which the disk is located.

Apertures 14 and 16 in disk 10 and balloon 12 are centrally situated therein. Disk 10 and balloon 12 are annular members each surrounding and defining the respective aperture.

Disk 10 and balloon 12 are made of a bioabsorbable biocompatible material. A surface 36 of disk 10 facing balloon 12 and/or a surface 38 of balloon 12 facing disk 10 is provided with a layer of a dormant adhesive substance that activated by the application of a predetermined form of energy, particularly a waveform energy such as electromagnetic radiation or ultrasonic vibration energy.

In a trans-organ surgical procedure as described in U.S. Pat. Nos. 5,297,536 and 5,458,131, a distal end portion of a surgical instrument is inserted through a natural body opening of a patient into a natural body cavity NBC (FIG. 3) of the patient. The surgical instrument is used to form a temporary artificial opening 40 through a wall 42 of an organ ORG defining the natural body cavity NBC. The surgical port device 8 described above with reference to FIGS. 1 and 2 is inserted through the natural body opening (mouth, anus, vaginal orifice, urethra) into the natural body cavity NBC (stomach, colon, vagina, urinary bladder). Subsequently, port device 8 is connected to wall 42 and disposed in artificial opening 40 to keep that opening open during a surgical procedure conducted via organ ORG and natural body cavity NBC, as described in U.S. Pat. Nos. 5,297,536 and 5,458,131.

The disposing of port device 8 includes inserting one disk 10 in a collapsed configuration through artificial opening 40 and expanding disk 10 and balloon 12 from collapsed configurations to expanded configurations so that the wall 42 of organ ORG is sandwiched between the expanded disk 10 and the expanded balloon 12, as shown in FIG. 3. Disk 10 and balloon 12 are connected to one another through artificial opening 40. Apertures 14 and 16 are aligned with opening 40 upon the disposing of port device 8 in opening 40. After the deployment of port device 8 in artificial opening 40, a distal end portion 44 of medical instrument 18 is inserted through the natural body opening (not shown), natural body cavity NBC, apertures 14 and 16, and artificial opening 40 into an internal space IS inside the patient.

Where port device 8 includes valve element or membrane 24, the inserting of the distal end portion 44 of medical instrument 18 includes engaging an outer surface of the medical instrument shaft 26 with the valve element 24 to form a seal about the medical instrument 18.

The surgical method further comprises introducing a pressurized gas into the internal space IS via tube 28. Tube 28 extends from a source of pressurized fluid such as carbon dioxide gas to internal space IS via port device 8.

As illustrated in FIG. 4, a port device 48 similar to port device 8 includes a disk 50 made of a flexible sheet material and a balloon 52. Disk 50 and balloon 52 define respective apertures 54 and 56 that are aligned with one another to define a hole (not separately designated) for the passage of a medical instrument 58 through the device. Balloon 52 is attached to disk 50 and has an inflation port (not shown) at the upstream end of an inflation tube 62 for enabling an introduction of a pressurizing fluid into the balloon to expand the balloon from a collapsed insertion configuration to an inflated use configuration.

At least one valve element 64 in the form of a self-sealing membrane or film is provided on the device for forming a seal about the shaft 66 of medical instrument 58 upon insertion thereof through apertures 54 and 56. Valve element or self-sealing membrane 64 may be realized as a resilient annular flange or film material about at least one of the apertures 54 and 56 in the disk 50 and the balloon 52. Thus, valve element 64 is attached at least one of the disk 50 and the balloon 52.

An elongate tube 68 is attached to disk 50 and balloon 52, the tube extending from a side of the disk opposite the balloon to a side of the balloon opposite the disk. In other words, tube 68 traverses the port device 48. This tube 68 is provided for the introduction of gas (e.g., carbon dioxide) to maintain pneumoperitoneum in the abdominal cavity (internal space) of a patient during a trans-organ procedure as described in U.S. Pat. Nos. 5,297,536 and 5,458,131. Accordingly, tube 68 is provided at one end with a port element (not shown) for coupling tube 68 to a source of pressurized gas (not shown). Tube 68 may extend through apertures 54 and 56 in disk 50 and balloon 52.

Disk 50 is provided along an edge or periphery 72 with a ring 74 of a resilient material stiffer than the flexible sheet material of the disk. Ring 74 assists in spreading disk 50 during a deployment procedure and maintaining the disk in an opened configuration against a wall 70 of internal body organ IBO during an intra-abdominal therapeutic or diagnostic procedure.

Apertures 54 and 56 in disk 50 and balloon 52 are centrally situated therein. Disk 50 and balloon 52 are annular members each surrounding and defining the respective aperture.

Disk 50 and balloon 52 are made of a bioabsorbable biocompatible material. A surface 76 of disk 50 facing balloon 52 and/or a surface 78 of balloon 52 facing disk 50 is provided with a layer of a dormant adhesive substance that activated by the application of a predetermined form of energy, particularly a waveform energy such as electromagnetic radiation or ultrasonic vibration energy. This energy is applied after the deployment of port device 48 in internal organ IBO, the insertion of balloon in a collapsed configuration through an artificial opening 80 in organ wall 70, the inflation of balloon 52, the expansion of disk 50 and the sandwiching of wall 70 between disk 50 and the inflated balloon 52.

Port device 48 is deployed and used as described above with reference to port device 8 and FIG. 3, except that balloon 52 rather than disk 50 is inserted in a collapsed configuration through the respective artificial opening 78.

Port device 48 may be distributed by itself or in a kit in combination with one or more medical instruments 44, including scalpels, forceps, scissors, cauterizers, snares, retrieval bags, etc.

Although the invention has been described in terms of particular embodiments and applications, one of ordinary skill in the art, in light of this teaching, can generate additional embodiments and modifications without departing from the spirit of or exceeding the scope of the claimed invention. For example, disk 10 and balloon 12 may be attached to a tubular member that forms a single through aperture or instrument passageway including aperture portions 14 and 16. Valve member or membrane 24 may then be located at any point along the tubular member. Accordingly, it is to be understood that the drawings and descriptions herein are profferred by way of example to facilitate comprehension of the invention and should not be construed to limit the scope thereof.

What is claimed is:

1. A surgical device comprising:
a disk made of a flexible sheet material; and
a balloon attached to said disk in parallel thereto in an expanded configuration of said disk and said balloon, so that said disk and said balloon define a through aperture for the passage of a medical instrument, said balloon having an inflation port for enabling an introduction of a fluid into said balloon to inflate same from a collapsed configuration to an inflated configuration.

2. The surgical device defined in claim 1, further comprising a valve element for forming a seal about an instrument shaft inserted through said aperture.

3. The surgical device defined in claim 2 wherein said valve element is a resilient annular flange.

4. The surgical device defined in claim 2 wherein said valve element is attached at least one of said disk and said balloon.

5. The surgical device defined in claim 1, further comprising an elongate tube attached to at least one of said disk and said balloon, said tube extending from a side of said balloon opposite said disk to a side of said disk opposite said balloon.

6. The surgical device defined in claim 5 wherein said tube extends through said aperture.

7. The surgical device defined in claim 5 wherein said tube is provided at one end with a port element for coupling said tube to a source of pressurized gas.

8. The surgical device defined in claim 1 wherein said disk is provided along an edge or periphery with a ring of a resilient material stiffer than the flexible sheet material of said disk.

9. The surgical device defined in claim 1 wherein said disk and said balloon surround said aperture.

10. The surgical device defined in claim 1 wherein a surface of at least one of said disk and said balloon is provided with a layer of an dormant adhesive substance that activated by the application of a predetermined form of energy.

11. The surgical device defined in claim 1 wherein said disk and said balloon are made of a bioabsorbable biocompatible material.

12. A surgical method comprising:
inserting a distal end portion of a surgical instrument through a natural body opening of a patient into a natural body cavity of the patient;
using said surgical instrument to form a temporary artificial opening through a wall of an organ defining said natural body cavity;
providing a surgical port device comprising a disk made of a flexible sheet material and a balloon, said disk and said balloon defining an aperture;
inserting said port device through said natural body opening into said natural body cavity;
subsequent to the inserting of said port device, attaching said port device to said wall of said organ in a region about in said artificial opening to keep the same open,
the disposing of said port device including inserting one of said disk and said balloon in a collapsed configuration through said artificial opening and expanding each of said disk and said balloon from a collapsed configuration to an expanded configuration so that said wall of said organ is sandwiched between the expanded disk and the expanded balloon and so that said disk and said balloon are connected to one another through said artificial opening,
said aperture being aligned with said artificial opening upon the disposing of said port device in said artificial opening; and
after the disposing of said port device in said artificial opening, inserting a distal end portion of a medical instrument through said natural body opening, said natural body cavity, said aperture, and said artificial opening into an internal space inside the patient.

13. The surgical method defined in claim 12 wherein said port device includes a valve element, the inserting of said distal end portion of said medical instrument including engaging an outer surface of said medical instrument with said valve element to form a seal about said medical instrument.

14. The surgical method defined in claim 12, further comprising introducing a pressurized gas into said internal space via an elongate tube communicating with said internal space via said port device.

15. The surgical method defined in claim 12 wherein said disk is provided along an edge or periphery with a ring of a resilient material stiffer than the flexible sheet material of said disk, the disposing of said port device including unfolding said ring from a folded configuration.

16. The surgical method defined in claim 12 wherein a selected one of said disk and said balloon is provided with a layer of an activatable adhesive, further comprising directing a predetermined form of energy towards a surface of said selected one of said disk and said balloon in contact with said wall of said organ to activate said adhesive.

17. A surgical kit comprising:
a surgical instrument having a distal end insertable through a natural body opening of a patient into a natural body cavity of the patient, said surgical instrument being provided with an operative tip utilizable to form a temporary artificial opening through a wall of an organ defining the patient's natural body cavity;
a surgical port device comprising a disk made of a flexible sheet material and a balloon connected to one another, said disk and said balloon defining an aperture, said port device being insertable through said natural body opening into said natural body cavity and subsequently attachable to the wall of the organ in a region about in the artificial opening to keep the same open,
said disk and said balloon being adapted to sandwich the wall of the organ between said disk and said balloon upon an expansion of said disk and said balloon so that said aperture is aligned with the artificial opening upon the disposing of said port device in the artificial opening; and
a medical instrument with a distal end portion through the natural body opening, the natural body cavity, said aperture, and the artificial opening into an internal space inside the patient.

18. The surgical kit defined in claim 17, further comprising an elongate tube for guiding a pressurized gas.

19. The surgical kit defined in claim 18 wherein said tube extends through said aperture.

20. The surgical kit defined in claim 17 wherein said port device includes a valve element disposed at said aperture.

21. The surgical kit defined in claim 17 wherein said disk is provided along an edge or periphery with a ring of a resilient material stiffer than the flexible sheet material of said disk.

22. The surgical kit defined in claim 17 wherein said one of said disk and said balloon is provided with a layer of an activatable adhesive.
